(19) United States
(12) Patent Application Publication
(10) Pub. No.: US 2022/0072031 A1
(43) Pub. Date: Mar. 10, 2022

(54) TREATMENT OF VIRAL INFECTION

(71) Applicant: Econugenics, Inc., SANTA ROSA, CA (US)

(72) Inventor: Isaac Eliaz, SANTA ROSA, CA (US)

(73) Assignee: Econugenics, Inc., SANTA ROSA, CA (US)

(21) Appl. No.: 17/063,505

(22) Filed: Oct. 5, 2020

Related U.S. Application Data
(63) Continuation-in-part of application No. 17/017,502, filed on Sep. 10, 2020.

(51) Int. Cl. A61K 31/732; A61K 45/06; A61K 31/375; A61K 31/59; A61K 39/42

(52) U.S. Cl. CPC A61K 31/732; A61K 45/06; A61K 2039/505; A61K 31/59; A61K 39/42; A61K 31/375

(57) ABSTRACT

The invention is directed to the treatment of COVID-19 patients by administering to patients either infected by SARS-CoV-2 or presymptomatic for COVID-19 but at risk of severe morbidity and mortality due to potential COVID-19 infection. Such patients are given modified pectin, preferably modified citrus pectin, in an amount that may range from 2.5 or 5 on up to 10-25 grams per day, divided into two or three doses per day. The administration may be oral, by inhalation, intrabuccal or IV. The process may be combined with the administration of supportive agents like antivirals, anti-inflammatories, immune based inhibitors, vitamins, monoclonal and polyclonal anti-viral or viral binding antibodies.

### PRIORITY DATA AND INCORPORATION BY REFERENCE

This application is related to, but does not claim priority from, U.S. Pat. Nos. 8,764,695; and 10,213,462; U.S. patent application Ser. No. 15/081,958 (allowed) and U.S. patent application Ser. No. 15/104,302. Each of these U.S. Patents and applications are incorporated herein-by-reference. Taken together, these patents clearly establish the efficacy of plasmapheresis to reduce the level in a mammal such as a human the level an agent that binds to a target also bound by circulating galectin-3 (gal-3 herein). This application is related to and claims priority from U.S. patent application Ser. No. 17/017,502 filed Sep. 10, 2020. The latter is directed principally to the treatment of SAR-CoV-2 infection by apheresis.

### BACKGROUND OF THE INVENTION

### Field of the Invention

This invention pertains to treatment of viral infections, which may include infections from MERS, SARS, SARS-CoV-2, INFLUENZA, MEASLES, HSV I&II, HPV and other opportunistic viruses. While this application focuses on SARS-CoV-2 and the disease caused thereby, COVID-19, treatment of other viruses and related viral agents may be affected by the same process. In late 2019, scientists in China first identified the emergence of a new virus. Sequencing of the genetic structure of that virus showed a strong morphological resemblance to other coronaviruses, and related viridae. Particular structural similarities to other coronavirus “spike proteins”—the proteins usually used by the virus to initiate cell penetration has been noted by those attempting to find a mechanism to treat and prevent the disease associated with infection by this coronavirus—COVID-19. See, e.g., Caniglia et al, Biochemistry, Biophysics & Molecular Biology, 2020 (pp. 1-10). Indeed, a structural review of the virus and its spike protein strongly suggests that some of the binding properties of the virus are related to those of gal-3—the structural similarities suggest that in fact the binding properties of the spike protein are not distinct from that of gal-3 itself. Revila et al, Frontiers in Immunology, August, 2020 (pp. 1-6).

This structural similarity is reinforced by the biological and pathological parallels between the virus, its disease, and the activities of properties of Gal-3 in the mammalian, and in particular, human body. Thus, as recounted in Caniglia et al, the pro-inflammatory response that is the signature of COVID-19 infection in the lungs of humans is a response activated by gal-3 (involving the release of IL-1b, IL-6 and TNF-α). The distribution of gal-3 in healthy humans reflects the distribution of distress in infected humans. The highest gal-3 in healthy individuals is in the lungs, followed by the gastrointestinal tract and then the brain. Remarkably, COVID-19 infected individuals exhibit predominantly symptoms of lung inflammation and fibrosis, followed by gastrointestinal symptoms (diarrhea, nausea and vomiting) followed, in turn, by neurological symptoms (cerebrovascular events, seizures, headaches and impaired consciousness). The striking parallel between gal-3 expression in the body, and COVID-19-induced gal-3 mediated events including inflammation, fibrosis, acute kidney injury and the like have led researchers to call for immediate exploration of gal-3 inhibitors as a possible treatment for COVID-19. Revilla et al—Hyperinflammation and Fibrosis in Severe COVID-19 Patients: Galectin-3, a Target Molecule to Consider (2020).

The strong relationship between the structure and activity of SARS-CoV-2 spike protein and the structure of gal-3 is echoed in the T cell response of those with COVID-19 who survive that infection. They exhibit a strong CD4 T cell response. A critical domain in the spike protein of β-coronaviridae is nearly identical in morphology to human Gal-3. The spike proteins are critical for the virus' entry into host cells, and its aggressive virulence. Gal-3 inhibition could be a path to a COVID-19 solution by a dual mechanism of reducing the host inflammatory immune response, T-cell activation, and interrupting viral attachment to host cells. A specific protocol for inhibiting gal-3 activity, and in particular, the activation and release mechanisms mediated by gal-3 specific to COVID-19, is not yet established. Establishing a safe and effective protocol for the administration of agents, be they inhibitors of gal-3 or other possible players in the “cytokine storm” phenomena that overwhelms so many COVID-19 patients, is a process of paramount importance. Developing new therapeutic agents and inhibitors, as well as appropriate dosages and protocols, may demand extended trials through Clinical III trials, while the population goes in need to an effective treatment. One treatment may be the apheresis process for treatment of COVID-19 set forth in U.S. patent application Ser. No. 17/017,502, filed Sep. 10, 2020. That application is incorporated herein-by-reference in its entirety, and benefit is claimed therefrom. In that application, apheresis is disclosed which may be effected by adsorbing COVID-19 by running it past or combining it with modified citrus pectin which binds to the spike proteins of the SARS-SoV-2 virus. The inventor herein has previously demonstrated the safe and effective administration of Pectasol-C, a well-studied composition of modified citrus pectin of molecular weight of about 2,000-25,000 Daltons in the context of treatment of conditions such as fibrosis and inflammation, as well as supporting immune response and other conditions. See, e.g., U.S. Pat. Nos. 9,427,449; 9,649,329; 10,555,966 and 8426567, all incorporated herein by-reference.

### SUMMARY OF THE INVENTION

The remarkable morphological and biological similarities between gal-3 and SARS-CoV-2, particularly the spike protein thereof, make it possible to take advantage of the well- established technology focused on the administration of a safe and effective agent to the infected patient and those likely to become infected. While Pectasol-C is a preferred form of Modified Citrus Pectin (MCP) for this invention, other compositions comprising modified pectin, including modified citrus pectin, prepared by enzymatic processes or related processes or of similar character and effect, may also be used. Studies indicate that treatment with a specific pH and heat-controlled enzymatic treatment to yield a product with a low molecular weight of <15 kilodaltons (kDa) and a degree of esterification under 5% yields an effective agent. Eliaz et al, Nutrients, (11) 2019. U.S. Pat. Nos. 6,274,566 and 6,462,029, incorporated herein by reference in their entirety, disclose the manufacture and use of compositions comprising low molecular weight modified pectins, including MCP and POS.

These patents particularly disclose the manufacture and use of chemically or enzymatically modified having an average molecular weight of 40,000 daltons or less. Low molecular weight pectins can be obtained from commercial sources, and methods for obtaining low molecular weight pectins are known in the art. See, e.g., Pienta et al., J. Nat'l Cancer Inst., 87: 348-52 (1995). The final modified pectin is preferably water soluble and has an average molecular weight of 40,000 daltons or less. Preferably, the modified pectin has an average molecular weight between about 10,000 daltons and about 20,000 daltons. More preferably, the modified alginate or modified pectin has an average molecular weight of about 10,000 daltons. The modified pectin can have a high degree of esterification, but preferably has a degree of esterification of less than about 10%. While it is possible to use MCP of greater molecular weight, administration by IV or modes other than oral required for such formulations deter patient compliance and complicate treatment. Where the patient requires it, or other co-administration requirements support it, administration can be inhalation, or intranasally and intrabuccal.

Agents may be co-administered with the MCP provided by this invention. This includes the administration of support compositions including ones established with some efficacy for COVID-19 such as antivirals (examples being Remdesivir, favipiravir and merimepodib); anti-inflammatories (Corticosteroids such as Dexamethasone, prednisone, methylprednisolone or hydrocortisone, and Nonsteroidal anti-inflammatory drugs (NSAIDs) such as Ibuprofen, Aspirin, Naproxen as well as Anti-inflammatory botanicals-like Padma Basic formula, quercetin, curcumin); immune based inhibitors (such as IL-6 inhibitors, TNF Alpha inhibitors); vitamin based therapy (such as vitamins C and D), and anti viral/viral binding monoclonal and polyclonal antibodies. Applicant also notes the MCP (or other gal-3 inhibitors) may play an essential role, by lowering SARS-CoV-2 levels and gal-3 levels. The modified pectin may be administered orally, iv, in inhalation, or intranasally and intrabuccally. Other regimens known to those of skill in the art, such as protocols for the protection and treatment of, e.g., kidney patients at risk may be appropriate. See, e.g., Gabarre et al, Intensive Care Med, 2020 and Shao et al, Pharmacological Research 161 (2020) (pp. 1-14) both of which are incorporated herein-by-reference for their discussion of conventional supportive treatments for COVID-19 patients with underlying kidney issues. These references discuss, for example, the conventional use of diuretics, acetylcysteine and sodium bicarbonate, which may be administered in conjunction with this invention. They will find greater efficacy if administered in conjunction with MCP as this agent has been shown to aid in reducing the damage due to the “cytokine storm” Many COVID-19 patients experience.

### DETAILED DESCRIPTION OF THE INVENTION

Particular attention has been focused on the development of critical kidney conditions aggravated or induced by COVID-19 infection. Although no standard treatments or practices have been developed, acute kidney injury (AM) has been observed in up to twenty-five percent of patients critically ill with COVID-19 infection. Gabarre et al, Intensive Care Med., (2020). Early detection of kidney injury in these patients, and specific therapy which may be supported by reducing gal-3 levels may be critical. In a survey of over twenty-four thousand COVID-19 patients, AKI was predictive of increased severe infection and mortality rates. Shao et al, Pharmacological Research (pp. 1-12) (2020). In the absence of a new standard protocol for treatment of AKI and chronic kidney infection related to COVID-19 infection, greater attention may be focused on controlling gal-3 levels together with existing treatments such as diuretics, acetylcysteine and sodium bicarbonate may be useful. In U.S. patent application Ser. No. 15/453,972 the administration of modified citrus pectin for the treatment of kidney injury is disclosed. That application is incorporated herein-by-reference. As administration of the same agent, albeit under shifted protocols and for different target populations, will be effective for COVID-19 patients who also are at risk for kidney injury, COVID-19 patients identified as at risk for kidney injury may particularly benefit from this invention.

Advantageously, this treatment with MCP administered to COVID-19 patients is combined with the administration of gal-3 blockers and inhibitors, and supportive treatments, such as those discussed above, including those such as disclosed in U.S. patent application Ser. No. 13/153,618. Although modified citrus pectin is a target inhibitor, other gal-3 inhibitors, such as other modified carbohydrates, including lactulosyl-l-leucine, Dermotte et al, Can. Res., 70 (19):476-88 (October 2010) as well as antibodies specific for gal-3, and other antagonists from very low molecular weight pectin weighing as low as 1KD to higher molecular weight products such as GCS-100, Streetly et al, Blood, 115(19):3939-48 (published Feb. 26, 2010 as an abstract) may be used. GCS is a polysaccharide derived from MCP, as opposed to reduced MCP. By removing large levels of plasma gal-3 from the blood, the disease, insult and injury due to inflammation or fibroses that is unfortunately commonly encountered in COVID-19 patients may be reduced, and the progression of the disease may be impeded. Similarly, conventional therapeutic treatments may be rendered more effective.

By significant reduction in circulating gal-3 levels in the presence of COVID-19 infection, inflammation and/or fibrosis can be controlled. In general, a reduction of circulating gal-3 of at least ten percent (10%) is necessary to achieve significant progress in gal-3 mediated fibroses, and even more may be required in acute conditions involving inflammation, fibroses due to viral infection. In functional terms, the reduction of gal-3 should be sufficient to reduce or inhibit the impact of gal-3 levels on inflammation and fibroses in said patient. Reduction in circulating gal-3 or blocking of circulating gal-3 of at least twenty percent (20%), and in some cases at least forty percent (40%) or even fifty percent (50%), may be required on a sustained basis. Severe situations, particularly critically ill COVID-19 patients experiencing their own cytokine storm may require reduction in circulating gal-3 levels in a mammalian patient of greater than fifty percent (50%) of that patient's circulating gal-3 titer, on up to seventy-five percent (75%) or even more. While some level of gal-3 in circulation is required for homeostasis, in acute situations, reductions at least by eighty percent (80%) of circulating gal-3, on up to near total removal of gal-3 from serum, may be called for, as that level is quickly replenished by the body.

The gal-3 levels in races other than Caucasians and subjects may vary, but regardless the target is to reduce or block gal-3 levels below the appropriate normal value. Viral particle reduction is the primary goal. This invention may be combined with apheresis to reduce gal-3 levels in patients in need of that reduction. If combined with gal-3 selective withdrawal, gal-3 target levels can vary based on the condition, age, gender, and other therapies involved.

Agents not specific to viral infection may be provided to the patient as part of the treatment of this invention as well. This includes a wide variety of active agents, but specifically includes agents such as chemotherapeutic drugs and therapeutic agents for the various conditions. For example, an anti-inflammatory will work better, cardiac medications, any drugs delivered to address an issue where COVID-19 infection and gal-3 are contributing factors, or prevent effective delivery to the target tissue, will be enhanced by this process. These agents will then have the opportunity to work under an environment of lower levels of gal-3. Even if just for a few hours, they can exhibit full biological activity. Once inflammation, for example, is reduced, naturally less gal-3 is being produced and expressed by the target tissue resulting in lower circulating gal-3 on a long-term basis. Thee agents can be administered orally, IV, IM, intranasal , in inhalation, when administered IV, the same IV access, post column, or different IV access can be used. They can be given during the apheresis, or shortly after.

Thus, the treatment addressed in this application is a treatment for a virally infected patient. Other viruses may present that can be addressed in similar fashion, but the virus particularly identified herein is SARS-CoV-2, one that causes COVID-19. The administration of MCP for this purpose may actually begin prior to detection of COVID-19 in a patient. A number of classes of highly susceptible potential patients, including the elderly, and those with underlying conditions like diabetes or kidney disease, individuals with heart ailments and similarly situated individuals. These patients are not only highly susceptible, but typically exhibit more sever morbidity and mortality once infected with COVID-19. Such patients are advantageously treated using this invention by administration of a preventative dosage of MCP on the order of 12-18 grams of MCP per day, more preferably 15 grams a day. However, different patient demands and the status and severity of infection will vary, patient to patient. Smaller doses as low as 5 gr/day or even 2.5 grams a day may still have benefit. This administration is most advantageously effected buy providing it in two or three divided dosages, such as 5 grams per dosage, three times a day. Other dosage protocols will be evident.

A symptomatic patient, that is, one exhibiting classic symptoms of COVID-19 infection, may be treated more aggressively following identification of the patient exhibiting COVID-19 symptoms, for example, with 15 up to 25 grams of MCP per day. Again, such dosages are advantageously distributed in two or three dosages per day, for example, 7-8 grams per day three times a day. In this respect, MCP has been demonstrated as safe, so such treatment levels do not pose issues of side-effects or deleterious downstream sequalae. Recovery from COVID-19 can be a long affair, depending on the patient. Convalescent patients are most effectively treated during convalescence by administration of MCP at lower dosages. Again, starting from a dosage level that may be as low as 2.5 or 5 grams a day, on up to larger doses of 15 grams of MCP per day in two or three dosages, the patient is slowly weaned to a maintenance level as the patient's symptoms improve. The term “an effective amount” is used herein to describe the dosage and treatment protocol that is required to impact the infection in a positive fashion. Applicant is aware of no negative side-effects or results associated with longer term administration of modified pectin. Accordingly, administration of maintenance doses of MCP, for example, 5 grams per day, so the maintenance administration of MCP may be administered on a long-term basis.

As noted, certain patients, particularly kidney patients with reduced eGFR values of under 20 may need to further reduce the amount of MCP administered due to issues of potassium clearance. Such patient may still be effectively treated by this invention. For patients with eGFR values about 20 or so, an MCP daily dosage of 10 grams per day may be appropriate. Those with very low values at or below 15 may benefit from a further reduction to 5 grams per day. Again, these lower dosages are still most effectively administered as distributed in a couple of different administrations each day.

While the present invention has been disclosed both generically, and with reference to specific alternatives, those alternatives are not intended to be limiting unless reflected in the claims set forth below. Variations that will immediately occur to those of skill in the art, include the co-administration of other well-established treatments in conjunction with modified pectins or MCP in particular. Alternate dosage protocols, and alternate classifications of presymptomatic individuals and “at risk” individuals will occur to those of skill in the art. Importantly, the COVID-19 pandemic has overtaxed critical care facilities and hospitals in particular. While the invention may rely on the diagnosis of a medical doctor for determination or identification of a COV ID-10 patient, the same is not required. Rapid assays to detect the formation of antibodies to SARS-CoV-2 in the blood have been developed, and an assay based on an individual's saliva sample is now available. Given that the administration of modified pectins, even modified citrus pectin of the type characterized, e.g., under forty thousand kilodaltons molecular weight, is not accompanied by known side-effects, this invention does not require the intervention of skilled medical individuals. The invention is limited only by the provisions of the claims, and their equivalents, as would be recognized by one of skill in the art to which this application is directed.

What is claimed is:

1. A method of treating a patient to address COVID-19 infection, comprising administering to said patient an effective amount of a daily dosage of modified pectin sufficient to prevent or treat COVID-19.

2. The method of claim 1 wherein said modified pectin is modified citrus pectin of a molecular weight under forty thousand Daltons.

3. The method of claim 1, wherein said dosage is at least 2.5 grams of modified pectin divided into two-three dosages per day.

4. A method of treating a patient at risk of severe morbidity or mortality associated with COVID-19 infection, comprising administering to said patient, prior to said patient exhibiting symptoms of COVID-19 infection, an effective amount of at least 12 grams of modified pectin per day, distributed in at least two doses per day.

5. The method of claim 1, wherein said dosage is from 15-30 grams of modified pectin per day, administered in 2-3 doses per day.

6. The method of claim 1, wherein said administration of modified pectin is accompanied by administration of at least one of anti-viral treatments, administration of anti-inflammatory treatments, administration of immune based, administration of at least one of vitamins C or D and administration of anti viral/viral binding monoclonal and polyclonal antibodies.

7. The method of claim 1, wherein said administration of modified pectin is continued until said patient is no longer at risk of developing COVID-19 illness associated complications.
